(19) United States
(12) Patent Application Publication
(10) Pub. No.: US 2006/0233736 A1
(43) Pub. Date: Oct. 19, 2006

(54) Cosmetic compositions containing mono-hydroxyl substituted amines and acid derivatives

(75) Inventors: Alan Meyers, Warren, NJ (US); Isabelle Hansenne, Westfield, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: L'OREAL, Paris (FR)

(21) Appl. No.: 11/108,298

(22) Filed: Apr. 18, 2005

(51) Int. Cl. A61K 8/55

(52) U.S. Cl. 424/70.23; 424/70.24

(57) ABSTRACT

Disclosed are cosmetic or dermatological compositions that include a mono-hydroxyl substituted amine such as dimethylaminoethanol (DMAE), a sulfonic, phosphoric, or phosphonic acid derivative, and a cosmetically acceptable carrier. Also disclosed are methods for making and using the compositions.

### BACKGROUND OF THE INVENTION

Soft, supple and flexible skin is cosmetically appealing and is an attribute of normal functioning epidermis. As human skin ages, however, the epidermis can become folded, rigid or furrowed to form wrinkles and sagging. Excessive exposure to sunlight may accelerate this transition. Exposure to cold or dry weather, detergents, solvents, and environmental pollutants may also lead to a loss of skin moisture, resulting in the loss of soft, supple and flexible skin.

Emollients such as fats, phospholipids and sterols have been used to soften wrinkled or dry skin. On the other hand, emollients do not provide protection from the sun, reduce sagging or the appearance of fine lines and wrinkles, or improve skin tone. There are a number of product compositions that target dry skin (U.S. Pat. Nos. 4,105,783 and 4,105,783), wrinkles (U.S. Pat. Nos. 4,424,234 and 5,091,171) and sun protection (U.S. Pat. No. 6,444,195).

Moisturizing agents, e.g., polyols, glycerin, and alpha-hydroxy acids, are present in many types of cosmetic products. Moisturizing agents provide hydration to the epidermis, but do not provide protection from harmful sun exposure. Conversely, sunscreens may block harmful sun rays, but generally do not improve the health or look of aging skin. Neither sunscreens nor moisturizing agents tends to improve aging skin tone or sagging.

### SUMMARY OF THE INVENTION

A first aspect of the present invention is directed to a cosmetic composition comprising a) a sulfonic acid derivative, a phosphoric acid derivative, or a phosphonic acid derivative, b) a mono-hydroxyl substituted amine, which in its non-protonated form, is represented by the formula:

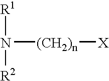

wherein R¹ and R², which may be same or different, each represents C₁-C₃ alkyl groups, X represents hydroxyl (OH) and n is 2, 3, 4, or 5, or a cosmetically or dermatological acceptable salt thereof, and c) a cosmetically acceptable carrier. In some embodiments, the compositions contain another active agent, e.g., sunscreens, anti-acne agents, anti-oxidants, carboxylic acids, and steroidal anti-inflammatory agents.

A second aspect of the present invention is directed to a method of treating e.g., moisturizing, conditioning and/or protecting, skin comprising applying to the skin a cosmetic composition comprising comprising a first active agent comprising a sulfonic acid derivative, a phosphoric acid derivative, or a phosphonic acid derivative; a mono-hydroxyl substituted amine, which in its non-protonated form, is represented by the formula:

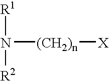

wherein R¹ and R², which may be same or different, each represents C₁-C₃ alkyl groups, X represents hydroxyl (OH) and n is 2, 3, 4, or 5; and a cosmetically acceptable carrier.

The compositions may improve the appearance and health of skin, including the promotion of a homogenous, clearer skin complexion, reduction of sagging, firming skin tone, reduction in skin pore size, the evening of skin texture, reduction of wrinkles, the treatment of acne and cutaneous scars, and otherwise provide a more attractive and youthful appearance.

### DETAILED DESCRIPTION

Broadly, the compositions of the present invention contain a mono-hydroxyl substituted amine, which in its non-protonated form, is represented by the formula: 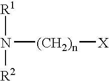
where R¹ and R², which may be same or different, each represents C₁-C₃ alkyl groups, X represents hydroxyl (OH) and n is 2, 3, 4, or 5. When R¹ and R² are methyl groups, and n is 2, the formula represents DMAE. DMAE may be prepared from equimolar amounts of ethylene oxide and dimethylamine. Merck Index 12: 2900 (1996). Alternatively, DMAE is commercially available from BASF under the tradename DMAE. Taken from the standpoint of DMAE, examples of cosmetically or dermatological acceptable salts may include DMAE acetylglumate, DMAE p-acetamidobenzoate, DMAE bitartrate, DMAE dihydrogen phosphate, DMAE hydrochloride, DMAE orotate, DMAE succinate, DMAE p-chlorophenoxyacetate hydrochloride, DMAE malonate and DMAE p-chlorophenoxyacetate. Other useful salts may include those derived from glycolic, citric and malic acids. Generally, the amine is present in the cosmetic compositions of the present invention in an amount of about 0.1% to about 5.0%, and preferably about 0.2% to about 3.0%, based on the total weight of the composition.

Sulfonic acid derivatives are known to stimulate desquamation of the skin, stimulate epidermal renewal, and help to combat aging skin (See, e.g., U.S. Patent Publication 2003/0232063). In some embodiments, the sulfonic acid derivative is represented by the formula: 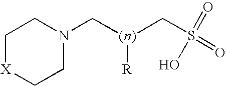
in which R is a hydrogen atom, —OH or NH₂; X is an oxygen atom, a group: 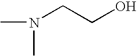
or a group: 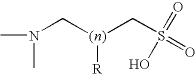
and n is 0, 1, 2, or 3. (See U.S. Patent Publication 2003/0232063.)

Sulfonic acid derivatives represented by the above formula are known essentially as organic buffers and are sold by companies such as Sigma, Aldrich or Fluka. In some embodiments, the first active agent comprising a sulfonic acid group is HEPES [4-(2-hydroxyethyl)piperazine-1-ethanesulfonic acid]. In other embodiments, the active agent represented by the above formula is 4-(2-hydroxyethyl)piperazine-1-(2-hydroxypropane-sulfonic acid), 4-(2-hydroxyethyl)piperazine-1-propanesulfonic acid, 3-morpholinopropanesulfonic acid, 2-morpholinoethanesulfonic acid, piperazine-1,4-bis(2-ethanesulfonic acid), and piperazine-1,4-bis(2-hydroxypropanesulfonic acid). In some other embodiments, the sulfonic acid derivative functions as a sunscreen. Examples of such derivatives include camphor derivatives such as 3-(4-methylbenzylidene)camphor or ((1,4-divinylbenzene)camphorsulfonic acid (also known as tetraphthalylidene dicamphor sulfonic acid), or derivatives thereof. See FR 2,528,420 and U.S. Pat. No. 4,585,597. The compound 1,4-divinylbenzene)camphorsulfonic acid is marketed by Chimex under the tradename Mexoryl SX. Other examples of sulfonic acid derivatives that are sunscreen agents include 2-phenylbenzimidazole-5-sulfonic acid and salts, 2-hydroxy-4-methoxybenzophenone-5-sulfonate, and (2-oxoborn-3-ylidene)-tolyl-4-sulfonic acid and soluble salts thereof.

Generally, the sulfonic acid derivative is present in the cosmetic compositions of the present invention in an amount of about 0.2% to about 30%, and preferably about 0.5% to about 20%, based on the total weight of the composition.

Phosphoric acid derivatives are known to function in cosmetic compositions surfactants (see, e.g., U.S. Pat. No. 6,733,765). Examples of such derivatives include monoalkyl and dialkyl esters of phosphoric acid and their salts, such as, for example, sodium mono- and dilauryl phosphate, potassium mono- and dilauryl phosphate, triethanolamine mono- and dilauryl phosphate, sodium mono- and dimyristyl phosphate, potassium mono- and dimyristyl phosphate, diethanolamine mono- and dimyristyl phosphate, triethanolamine mono- and dimyristyl phosphate, the potassium salt of dodecylphosphoric acid, as a mixture of monoester and diester (mainly diester), sold under the name Crafol AP-31™ by Cognis, the mixture of monoester and diester of octylphosphoric acid, sold under the name Crafol AP-20™ by Cognis, the mixture of ethoxylated (7 mol of EO) phosphoric acid monoester and diester of 2-butyloctanol, sold under the name Isofol 12 7 EO-Phosphate Ester™ by Condea. See, e.g., U.S. Pat. No. 6,733,765. In other embodiments, the phosphoric acid derivative is a lethicin, which is a mixture of phospholipids, i.e., of diglycerides of fatty acids linked to an ester of phosphoric acid. Preferably, lecithins are diglycerides of stearic, palmitic, and oleic acids linked to the choline ester of phosphoric acid. See, e.g., U.S. Pat. No. 6,558,697.

In embodiments wherein the cosmetic composition is in the form of an emulsion, the phosphoric acid derivative may be an anionic emulsifier that is liquid at room temperature, i.e., at a temperature ranging from 15° C. to 25° C. It can be chosen in particular from the group of anionic surfactants containing a phosphate group, which are liquid at room temperature, such as mono-, di- and/or triesters of phosphoric acid and of a C₁₂ to C₂₂ fatty alcohol, i.e., of an alcohol having an alkyl chain containing from 12 to 22 carbon atoms (inclusive of all specific values and subranges therebetween, such as 14, 16, 18 and 20 carbon atoms), and mono-, di- and/or triesters of phosphoric acid and of an ethoxylated fatty alcohol, having a fatty chain containing from 12 to 22 carbon atoms (inclusive of all specific values and subranges therebetween, such as 14, 16 from 20 carbon atoms) and a number of ethoxylated units ranging from 1 to 100 and preferably from 4 to 25 (inclusive of all specific values and subranges therebetween, such as 2, 5, 10, 15, 35, 50, 75 and 90). Examples of such anionic emulsifiers that are liquid at room temperature include oleyl phosphate (mixture of mono- and diester of phosphoric acid and of oleyl alcohol), trioleyl phosphate, dilaureth-4 phosphate (mixture of diesters of phosphoric acid and of polyethylene glycol ether and of lauryl alcohol, containing 4 oxyethylenated groups), trioleth-8 phosphate (mixture of triesters of phosphoric acid and of ether of polyethylene glycol and of oleyl alcohol, containing 8 oxyethylenated groups), triceteareth-4 phosphate (mixture of triesters of phosphoric acid and of ether of polyethylene glycol and of cetyl and stearyl alcohols, containing 4 oxyethylenated groups), the monoester of phosphoric acid and of stearic and isostearic acid (CTFA name: octyldecyl phosphate) sold under the name Hostaphat CG 120 by Clariant, and mixtures thereof. See, e.g., U.S. Pat. No. 6,358,158. In the case of nanoemulsions, phosphoric acid fatty esters and their oxyethylenated derivatives may be useful. See, e.g., U.S. Pat. No. 6,274,150.

Generally, a phosphoric acid derivative is present in the cosmetic compositions of the present invention in an amount about 0.05% to about 10%, and preferably about 0.075% to about 5%, based on the total weight of the composition.

Phosphonic acid derivatives may be useful to promote adhesion to keratinous substances (such as the phosphonic acid silicon alkoxides disclosed in U.S. Pat. No. 6,607,713). They may also act as metal chelating agents, sequestering agents and as stabilizers in acidic solutions. In some embodiments, the phosphonic acid derivative is 1-hydroxy-ethylidene 1,1-diphosphonic acid, aminotri(methylene phosphonic)acid, hexamethylenediaminetetra(methylenephosphonic)acid, diethylenetriamine pentamethylene phosphonic acid; diethylenetriamine tetramethylene phosphonic acid; or ethylenediamine tetramethylene phosphonic acid, and their salts, e.g., ammonium and alkaline metal salts, e.g., sodium salts. Representative salts may include the heptasodium salt of diethylenetriamine pentamethylene phosphonic acid, the pentasodium salt of diethylenetriamine tetramethylene phosphonic acid, and the pentasodium salt of ethylenediamine tetramethylene phosphonic acid. Ethylenediaminetetra(methylenephosphonic)acid is sold by the Monsanto company under the name Dequest 2041, and the pentasodium salt of this acid is sold by Monsanto under the name Dequest 2046. (See U.S. Pat. Nos. 5,352,438; 5,552,446; 6,337,077 and 6,465,510.)

Generally, the phosphonic acid derivative is present in the cosmetic compositions of the present invention in an amount of about 0.05% to about 10%, and preferably about 0.075% to about 5.0%, based on the total weight of the composition.

In general, the ratio of the amount of DMAE to the amount of sulfonic, phosphoric or phosphonic acid derivative is about 0.003 to about 60, and preferably about 0.1 to about 10.

The nature of the carrier depends upon the form of the composition. The compositions according to the present invention can be provided in various forms, principally in the form of emulsions (e.g., creams), gels and lotions. They may also be in anhydrous form e.g., in the form of an ointment, gel or pomade. Emulsions and gels are preferred. Compositions of the present invention other than those in anhydrous form have an aqueous phase and an oil or fatty phase. When the composition according to the invention is an emulsion, the proportion of the fatty phase generally ranges from about 0.5% to about 80% by weight and preferably from about 5% to about 50% by weight, based on the total weight of the composition. Oils, waxes, emulsifiers and co-emulsifiers that are typically present in the composition may be selected from those conventionally used in cosmetics and dermatology.

The fatty phase usually contains at least one oil. Examples include hydrocarbon-based oils of animal origin, such as perhydrosqualene; hydrocarbon-based oils of plant origin, such as liquid triglycerides of fatty acids containing from 4 to 10 carbon atoms, for instance heptanoic or octanoic acid triglycerides or alternatively, for example, sunflower oil, corn oil, soybean oil, marrow oil, grapeseed oil, sesame oil, hazelnut oil, apricot oil, macadamia oil, arara oil, sunflower oil, castor oil, avocado oil, caprylic/capric acid triglycerides, jojoba oil or karite butter oil; synthetic esters and synthetic ethers, especially of fatty acids, for instance oils of formulae R6COOR7 and R6OR7, in which R6 represents a fatty acid residue containing from 8 to 29 carbon atoms, and R7 represents a branched or unbranched hydrocarbon-based chain containing from 3 to 30 carbon atoms, such as, for example, purcellin oil, isononyl isononanoate, isopropyl myristate, 2-ethylhexyl palmitate, 2-octyl-dodecyl stearate, 2-octyldodecyl erucate or isostearyl isostearate; hydroxylated esters such as isostearyl lactate, octyl hydroxystearate, octyldodecyl hydroxystearate, diisostearyl malate, triisocetyl citrate and fatty alkyl heptanoates, octanoates and decanoates; polyol esters such as propylene glycol dioctanoate, neopentyl glycol diheptanoate and diethylene glycol diisononanoate; and pentaerythritol esters such as pentaerythrityl tetraisostearate; linear or branched hydrocarbons of mineral or synthetic origin, such as volatile or non-volatile liquid paraffins, and derivatives thereof, petroleum jelly, polydecenes, and hydrogenated polyisobutene such as parleam oil; fatty alcohols containing from 8 to 26 carbon atoms, such as cetyl alcohol, stearyl alcohol and a mixture thereof (cetylstearyl alcohol), octyldodecanol, 2-butyloctanol, 2-hexyldecanol, 2-undecylpentadecanol, oleyl alcohol or linoleyl alcohol; alkoxylated and ethoxylated fatty alcohols such as oleth-12; partially hydrocarbon-based and/or silicone-based fluoro oils such as perfluoromethylcyclopentane, perfluoro-1,3-dimethylcyclohexane, perfluoro-1,2-dimethylcyclo-butane; perfluoroalkanes such as dodecafluoropentane and tetradecafluorohexane, bromoperfluorocaprylyl bromide; nonafluoromethoxybutane and nonafluoro-ethoxyisobutane; perfluoromorpholine derivatives, such as 4-trifluoromethylperfluoromorpholine; silicone oils such as volatile or non-volatile polymethylsiloxanes (PDMSs) containing a linear or cyclic silicone chain, and that are liquid or pasty at room temperature, e.g., cyclopolydimethyl-siloxanes (cyclomethicones) such as cyclohexa-siloxane; polydimethylsiloxanes comprising alkyl, alkoxy or phenyl groups, that are pendent or at the end of a silicone chain, these groups containing from 2 to 24 carbon atoms; phenylsilicones, such as phenyltrimethicones, phenyldimethicones, phenyltrimethylsiloxydiphenylsiloxanes, diphenyldimethicones, diphenylmethyldiphenyltrisiloxanes, 2-phenylethyl-trimethylsiloxysilicates and polymethylphenyl-siloxanes; and mixtures thereof.

Other substances that may be present in the oily phase are, for example, fatty acids containing from 8 to 30 carbon atoms, such as stearic acid, lauric acid, palmitic acid and oleic acid; waxes of animal origin, such as lanolin, beeswax, spermaceti or lanolin derivatives, such as lanolin alcohols, hydrogenated, hydroxylated or acetylated lanolin, lanolin fatty acids and acetylated lanolin alcohol; waxes of vegetable origin, such as carnauba, candelilla, kapok, ouricury, rice, hydrogenated jojoba, esparto or japan wax or cork fibre or sugar cane waxes or cocoa butter; mineral waxes, for example paraffin, montan, lignite or petrolatum waxes or microcrystalline waxes, ceresin or ozokerite; or synthetic waxes, such as polyethylene waxes, waxes obtained by the Fischer-Tropsch synthesis and linear esters resulting from the reaction of a saturated C10 to C40 carboxylic acid and of a saturated C10 to C40 alcohol, such as myristyl myristate. Use may also be made of cetyl alcohol, stearyl alcohol, calcium lanolates or stearates, castor oil, palm oil, coconut oil, sunflower oil or hydrogenated coconut oil; gums such as silicone gums (dimethiconol); silicone resins such as trifluoromethyl-C1-4-alkyldimethicone and trifluoro-propyldimethicone; and silicone elastomers. These fatty substances may be chosen in a varied manner to prepare a composition having the desired properties, e.g., consistency or texture.

Emulsions may contain at least one emulsifier e.g., amphoteric, anionic, cationic and nonionic emulsifiers, used alone or as a mixture. Choice of emulsifier depends upon the nature of the emulsion e.g., water-in-oil (W/O) or oil-in-water (O/W) emulsions. Examples of emulsifiers that may be used in O/W emulsions include nonionic emulsifiers such as saccharide esters and ethers such as sucrose stearate, sucrose cocoate (and mixtures of sucrose stearate and cocoate); polyol esters, in particular glycerol or sorbitol esters, such as glyceryl stearate, polyglyceryl-2 stearate and sorbitan stearate; glycerol ethers; oxyethylenated and/or oxypropylenated ethers such as the oxyethylenated, oxypropylenated ether of lauryl alcohol containing 25 oxyethylene groups and 25 oxypropylene groups (CTFA name “PPG-25 laureth-25”) and the oxyethylenated ether of the mixture of C12-C15 fatty alcohols containing 7 oxyethylene groups (CTFA name “C12-C15 Pareth-7”); ethylene glycol polymers such as PEG-100, and mixtures thereof.

Examples of emulsifiers for use in W/O emulsions include fatty esters of a polyol, in particular of glycerol or of sorbitol, and in particular polyol isostearates, oleates and ricinoleates; saccharide esters and ethers such as methyl glucose dioleate; fatty esters such as magnesium lanolate; dimethicone copolyols and alkyldimethicone copolyols.

Examples of surfactants (emulsifying and coemulsifying) include the esters of fatty acids and polyethylene glycol (PEG), esters of fatty acids and glycerol (glyceryl stearate) or esters of fatty acids and sugar (sorbitan stearate), as well as the polyoxyethylenated or polyoxypropylenated derivatives thereof, cyclomethicones and dimethicone copolyols, and also anionic surfactants (e.g., potassium or sodium alkyl phosphates).

Gels are obtained using gelling or thickening agents. Gelling or thickening agents that may be mentioned include, for example, carboxyvinyl polymers, e.g., crosslinked acrylates (e.g. Carbopol 982), hydrophobically-modified acrylates (e.g. Carbopol 1382); polyacrylamides such as, for example, the crosslinked copolymers sold under the names Sepigel 305 (CTFA name: polyacrylamide/C13-C14 isoparaffin/Laureth 7) or Simulgel 600 (CTFA name: acrylamide/sodium acryloyldimethyltaurate copolymer/isohexadecane/polysorbate 80) by SEPPIC; 2-acrylamido-2-methylpropanesulphonic acid polymers and copolymers, that are optionally crosslinked and/or neutralized, for instance the poly(2-acrylamido-2-methylpropanesulphonic acid) sold by the company Hoechst under the trade name “Hostacerin AMPS” (CTFA name: ammonium polyacryldimethyltauramide); cellulose derivatives such as hydroxyethylcellulose, sodium carboxymethylcellulose, hydroxypropyl methylcellulose, hydroxypropyl cellulose, ethyl cellulose and hydroxymethyl cellulose; polysaccharides and natural and synthetic gums, e.g., natural gums such as xanthan gum, sclerotium, carrageenan and pectin; and mixtures thereof, modified clays (bentones), metal salts of fatty acids (aluminum stearate), ethylene/acrylate copolymers, silicas and polyethylenes. Amounts of the gelling agent or thickener generally range from about 0.0001% to about 15%. In some embodiments, the amount ranges from about 0.001% to about 1%, from about 0.01% to about 0.5%, or from about 0.5 to about 15%, all based on total weight of the cosmetic composition.

Lotions generally contain the active agent(s), solubilized in an oil phase and/or the water phase depending on solubility of the agent(s). The ointments are anhydrous compositions based, for example, on petrolatum, paraffin oil and/or waxes.

The cosmetic compositions of the present invention may further comprise one or more cosmetic agents or dermatological active agents e.g., agents capable of treating or preventing any sign of aging of the skin. The active agents may be chosen, for example, from moisturizers, free-radical scavengers, keratolytic agents, vitamins, anti-elastase and anti-collagenase agents, peptides, fatty acid derivatives, steroids, trace elements, bleaching agents, extracts of algae and of planktons, sunscreens, enzymes and coenzymes, flavonoids and ceramides, α-hydroxy acids and mixtures thereof.

Useful moisturizers include sodium lactate; polyols, and in particular glycerol, sorbitol and polyethylene glycols; mannitol; amino acids; hyaluronic acid; lanolin; urea and mixtures containing urea, such as NMF (“Natural Moisturizing Factor”); petroleum jelly; and mixtures thereof.

Useful free-radical scavengers include ethylenediaminetetraacetic acid and its salts such as sodium salt; guanosine; superoxydismutase; tocopherol (vitamin E) and its derivatives (acetate); ethoxyquine; lactoferrin; lactoperoxidase and nitroxide derivatives; superoxide dismutases; glutathione peroxidase; plant extracts with free-radical-scavenging activity, such as the aqueous extract of wheat germ sold by the company Silab under the name Detoxiline; and mixtures thereof.

Useful keratolytic agents include α-hydroxy acids, especially acids derived from fruit, for instance glycolic acid, lactic acid, malic acid, citric acid, tartaric acid and mandelic acid, derivatives thereof and mixtures thereof; β-hydroxy acids, for instance salicylic acid and its derivatives such as 5-n-octanoylsalicylic acid or 5-n-dodecanoylsalicylic acid; α-keto acids, for instance ascorbic acid or vitamin C and its derivatives such as its salts, for instance sodium ascorbate, magnesium ascorbyl phosphate or sodium ascorbyl phosphate; its esters, for instance ascorbyl acetate, ascorbyl palmitate and ascorbyl propionate, or its sugars, for instance glycosylated ascorbic acid, and mixtures thereof; β-keto acids; retinoids, for instance retinol (vitamin A) and its esters, retinal, retinoic acid and its derivatives, and also the retinoids described in documents FR-A-2,570,377, EP-A-199 636, EP-A-325-540 and EP-A-402 072; and mixtures thereof.

Useful vitamins, in addition to vitamins A, E and C indicated above, include vitamin B3 (or vitamin PP or niacinamide), vitamin B5 (or panthenol), vitamin D, vitamin F, derivatives, analogues and precursors of these vitamins and also those of vitamins A, E and C, for instance lycopenes or carotenes that are precursors of vitamin A, and mixtures thereof. Vitamin B3 derivatives include nicotinic acid esters, such as tocopherol nicotinate; amides derived from niacinamide by substitution of the hydrogen groups of —CONH₂; products of reaction with carboxylic acids and amino acids; esters of nicotinyl alcohol and of carboxylic acids such as acetic acid, salicylic acid, glycolic acid or palmitic acid. Mention may also be made of the following derivatives: 2-chloronicotinamide, 6-methylnicotinamide, 6-aminonicotinamide, N-methylnicotinamide, N,N-dimethylnicotinamide, N-(hydroxymethyl)nicotinamide, quinolinic acid imide, nicotinanilide, N-benzylnicotinamide, N-ethylnicotinamide, nifenazone, nicotinaldehyde, isonicotinic acid, methylisonicotinic acid, thionicotinamide, nialamide, 2-mercaptonicotinic acid, nicomol and niaprazine. Other vitamin B3 derivatives which may also be mentioned include its mineral salts such as the chlorides, bromides, iodides and carbonates, and its organic salts, such as the salts obtained by reaction with carboxylic acids such as acetate, salicylate, glycolate, lactate, malate, citrate, mandelate and tartrate.

As vitamin B5, it is also possible to use panthenol or panthenyl alcohol or 2,4-dihydroxy-N(3-hydroxypropyl)-3,3-dimethylbutanamide, in its various forms: D-panthenol, DL-panthenol, and its derivatives and analogues, such as calcium pantothenate, pantethine, pantotheine, ethyl panthenyl ether, pangamic acid, pyridoxine and pantoyllactose, and natural compounds containing them such as royal jelly.

As vitamin D, mention may be made of 1α, 25-dihydroxy vitamin D3 and its analogues, and also vitamin D analogues, such as those described in document WO-A-00/26167, such as, for example: 3-hydroxymethyl-5-{2-[3-(5-hydroxy-5- or 6-methylhexyl)-phenyl]-vinyl}-phenol, 3-[3-(5-hydroxy-1,5-dimethyl-hexyl)-phenoxymethyl]-5-hydroxymethyl-phenol, 6-[3-(3,4-bis-hydroxymethyl-benzyloxy)-phenyl]-2-methyl-hepta-3,5-dien-2-ol, 6-[3-(3,4-bis-hydroxymethyl-benzyloxy)-phenyl]-2-methyl-hexan-2-ol, 6-[3-(3,4-bis-hydroxymethyl-phenoxymethyl)-phenyl]-2-methyl-heptan-2-ol, 7-[3-(3,4-bis-hydroxymethyl-phenoxymethyl)-phenyl]-3-ethyl-octan-3-ol, 5-{2-[4-(5-hydroxy-5-methyl-hexyl)-phenyl]-vinyl or -ethyl}-benzene-1,3-diol, 5-(2-[3- or 4-(6-hydroxy-6-methyl-heptyl)phenyl]vinyl}-benzene-1,3-diol, 5-{2-[3- or 4-(6-hydroxy-6-methyl-heptyl)-phenyl]ethyl-benzene-1,3-diol, 2-hydroxymethyl-4-{2-[3- or 4-(5-hydroxy-5-methylhexyl)-phenyl]-vinyl-phenol, 2-hydroxymethyl-4-{2-[3- or 4-(6-hydroxy-6-methylheptyl)-phenyl]-vinyl}-phenol, 2-hydroxymethyl-4-{2-[3- or 4-(5-hydroxy-5-methylheptyl)-phenyl]-ethyl}-phenol, 2-hydroxymethyl-4-{2-[3- or 4-(6-hydroxy-6-methylheptyl)-phenyl]-ethyl}-phenol, 2-hydroxymethyl-5-(2-[4-(5-hydroxy-5-methyl-hexyl)phenyl]-vinyl-phenol, 6-[3-(3,4-bis-hydroxymethyl-benzyloxy)-phenyl-1,2-methyl-heptan-2-ol, 4-[3-(5-hydroxy-1,5-dimethyl-hexyl)-phenoxymethyl]2-hydroxymethyl-p-phenol, 6-[3- or 4-[2-(3,4-bis-hydroxymethyl-phenyl)-vinyl]phenyl}-2-methyl-hexan-2-ol, 7-{4-[2-(3,4-bis-hydroxymethyl-phenyl)-vinyl]phenyl}-2-methyl-heptan-2-ol, 5-(2-[3-(6-hydroxy-6-methyl-heptyl)-phenyl]-1-methylvinyl-benzene-1,3-diol, 5-{2-[3-(5-hydroxy-5-methyl-hexyl)-phenyl]-vinyl}benzene-1,3-diol, 5-[3-(6-hydroxy-6-methyl-heptyl)-phenoxymethyl]benzene-1,3-diol, 5-{2-[3-(7-hydroxy-7-methyl-oct-1-enyl)-phenyl]vinyl}-benzene-1,3-diol, 5-{2-[3-(7-hydroxy-7-methyl-octyl)-phenyl]-vinyllbenzene-1,3-diol, 4-{2-[3-(6-hydroxy-6-methyl-heptyl)-phenyl]-vinyl)benzene-1,2-diol, 3-{2-[3-(6-hydroxy-6-methyl-heptyl)-phenyl]-vinyl}phenol, 6-{3-[2-(3,5-bis-hydroxymethyl-phenyl)-vinyl]phenyl}-2-methyl-hexan-2-ol, 3-{2-[3-(7-hydroxy-7-methyl-octyl)-phenyl]-vinyl}phenol, 7-{3-[2-(3,5-bis-hydroxymethyl-phenyl)-vinyl]phenyl-2-methyl-heptan-2-ol, 7-{3-[2-(3,4-bis-hydroxymethyl-phenyl)-vinyl]phenyl}-2-methyl-heptan-2-ol, 7-{3-[2-(4-hydroxymethyl-phenyl)-vinyl]-phenyl}2-methyl-heptan-2-ol-, 4-{2-[3-(7-hydroxy-7-methyl-oct-1-enyl)-phenyl]vinyl)-benzene-1,2-diol, 7-[3-(3,4-bis-hydroxymethyl-phenylethynyl)-phenyl]2-methyl-heptan-2-ol, 5-{2-[3-(6-hydroxy-6-methyl-hept-1-enyl)-phenyl]vinyl}-benzene-1,3-diol, 5-{2-[3-(7-ethyl-7-hydroxy-non-1-enyl)-phenyl]vinyl)-benzene-1,3-diol, 5-{2-[3-(7-hydroxy-1-methoxy-1,7-dimethyl-octyl)phenyl]-vinyl-benzene-1,3-diol, 5-{2-[3-(6-hydroxy-1-methoxy-1,6-dimethyl-heptyl)phenyl]-vinyl}-benzene-1,3-diol, 5-{2-[3-(5-hydroxy-pentyl)-phenyl]-vinyl-benzene-1,3-diol, 5-{2-[3-(5-hydroxy-6-methyl-heptyl)-phenyl]-vinyl}benzene-1,3-diol, 5-{2-[3-(6-hydroxy-7-methyl-octyl)-phenyl]-vinyl)benzene-1,3-diol, 5-{2-[3-(5-hydroxy-6-methyl-hept-1-enyl)-phenyl]vinyl}-benzene-1,3-diol, 5-{2-[3-(6-hydroxy-7-methyl-oct-1-enyl)-phenyl]vinyl}-benzene-1,3-diol, 5-{2-[3-(1,6-dihydroxy-1,6-dimethyl-heptyl)-phenyl]vinyl}-benzene-1,3-diol, 5-(2-[3-(6-hydroxy-1,6-dimethyl-hept-1-enyl)-phenyl]vinyl}-benzene-1,3-diol.

Vitamin F is a mixture of essential fatty acids, that is to say of unsaturated acids containing at least one double bond, such as linoleic acid or 9,12-octadecadienoic acid, and its stereoisomers, linolenic acid in alpha form (9,12,15-octadecatrienoic acid) or the gamma form (6,9,12-octadecatrienoic acid) and stereoisomers thereof, arachidonic acid or 5,8,11,14-eicosatetraenoic acid and its stereoisomers.

Vitamin F or analogues thereof such as mixtures of unsaturated acids containing at least one double bond and especially mixtures of linoleic acid, of linolenic acid and of arachidonic acid, or compounds containing them and especially oils of plant origin containing them such as, for example, jojoba oil, may be used in the compositions of the present invention.

Useful anti-elastase agents include peptide derivatives and especially peptides from leguminous seeds such as those sold by Laboratoires Seriobiologiques de Nancy under the reference Parelastyl; the N-acylamino amide derivatives described in patent application FR-A-2,180,033, such as, for example, ethyl {2-[acetyl(3-trifluoromethylphenyl)amino]-3-methylbutyrylamino}acetate and (2-[acetyl-(3-trifluoromethylphenyl)amino]-3-methylbutyrylamino]acetic acid, and mixtures thereof. Anti-collagenase agents that may be mentioned include metalloprotease inhibitors, such as ethylenediamine acid (EDTA) and cysteine, and mixtures thereof.

Useful peptides include proteins (wheat or soybean protein), hydrolysates thereof, for instance those sold by the company Silab under the name Tensine, and mixtures thereof.

Useful fatty acid derivatives include polyunsaturated phospholipids including the essential fatty acid phospholipids from octopus, and mixtures thereof.

Useful steroids include DHEA or dehydroepiandrosterone, its biological precursors, its metabolites, and mixtures thereof. The expression “biological precursors” of DHEA especially means A5-pregnenolone, 17α-hydroxypregnenolone and 17α-hydroxypregnenolone sulphate. The expression “DHEA derivatives” means both its metabolic derivatives and its chemical derivatives. Metabolic derivatives that may especially be mentioned include Δ5-androstene-3,17-diol and especially 5-androstene-3β,17β-diol, A4-androstene-3,17-dione, 7-hydroxy DHEA (7α-hydroxy DHEA or 7β-hydroxy DHEA) and 7-keto-DHEA which is itself a metabolite of 7β-hydroxy DHEA. A preferred group is dehydroepiandrosterone, 5-pregnenolone, 17-hydroxypregnenolone, 17-hydroxypregnenolone sulphate, 5-androstene-3,17-diol, 4-androstene-3,17-dione, 7-hydroxy DHEA, 7-hydroxy DHEA, 7-keto-DHEA, and mixtures thereof.

Useful trace elements include copper, zinc, selenium, iron, magnesium and manganese, and mixtures thereof.

Useful bleaching agents include any compound for treating or preventing age marks, that is to say any depigmenting compound which acts directly on the vitality of the epidermal melanocytes in which melanogenesis takes place and/or which interferes with one of the steps in the biosynthesis of melanin either by inhibiting one of the enzymes involved in melanogenesis or by becoming intercalated as a structural analogue of one of the chemical compounds in the synthetic chain of melanin, which chain may thus be blocked and bring about the depigmentation. Bleaching active agents that may be mentioned, for example, include kojic acid and its derivatives, hydroquinone and its derivatives such as arbutin and its esters; ellagic acid and its derivatives; plant extracts, and especially extracts of licorice, of mulberry or of scutellaria; glutathione and its precursors; cysteine and its precursors; the compounds derived from aminophenol that are described in document WO-A-99/10318, such as, especially, N-ethyloxycarbonyl-4-aminophenol, N-ethyloxycarbonyl-O-ethyloxycarbonyl-4-aminophenol, N-cholesteryloxycarbonyl-4-aminophenol and N-ethylaminocarbonyl-4-aminophenol; and mixtures of these compounds.

Useful extracts of algae include extracts of red or brown algae and, for example, the extract of brown algae from the Laminaria family, for instance the extracts from the species Laminaria digitata, and more particularly the product sold by the company CODIF under the name Phycosaccharides, which is a concentrated solution of an oligosaccharide obtained by controlled enzymatic depolymerization of membrane polysaccharides of a brown alga. It comprises a sequence of two uric acids, namely mannuronic acid and guluronic acid.

Useful extracts of planktons include plankton in aqueous dispersion (CTFA name: Vitreoscilla Ferment) sold under the name Mexoryl SAH by the company Chimex.

The compositions of the present invention may also comprise sunscreens, in addition to the sulfonic acid derivatives that function as such. Such a second active agent sunscreen may be chemical absorbers that actually absorb harmful ultraviolet radiation. It is well known that chemical absorbers are classified as either UV-A or UV-B absorbers, depending on the type of radiation they protect against. UV-A absorbers generally absorb radiation in the 320 to 400 nm region of the ultraviolet spectrum. UV-A absorbers include anthranilates, benzophenones, and dibenzoyl methanes. UV-B absorbers generally absorb radiation in the 280 to 320 nm region of the ultraviolet spectrum. UV-B absorbers include p-aminobenzoic acid derivatives, cinnamates, and salicylates.

Classifying the chemical absorbers generally as UV-A or UV-B absorbers is accepted within the industry. However, a more precise classification is one based upon the chemical properties of the sunscreens. There are eight major classifications of sunscreen chemical properties that are discussed at length in “Sunscreens—Development, Evaluation and Regulatory Aspects,” by N. Shaath et al., 2nd. Edition, pages 269-273, Marcel Dekker, Inc. (1997).

The sunscreens useful in the present invention typically comprise chemical absorbers, but may also comprise physical blockers. Exemplary sunscreens which may be formulated into the compositions of the present invention are chemical absorbers such as p-aminobenzoic acid derivatives, anthranilates, benzophenones, cinnamic derivatives, dibenzoyl methanes (such as avobenzone also known as Parsol® 1789), diphenylacrylate derivatives, salicylic derivatives, triazine derivatives, benzimidazole compounds, bis-benzoazolyl derivatives, methylene bis-(hydroxyphenylbenzotriazole) compounds, the sunscreen polymers and silicones, or mixtures thereof. These are variously described in U.S. Pat. Nos. 2,463,264, 4,367,390, 5,166,355 and 5,237,071 and in EP 863,145, EP 517,104, EP 570,838, EP 796,851, EP 775,698, EP 878,469, EP 933,376, EP 893,119, EP 669,323, GB 2,303,549, DE 1,972,184 and WO 93/04665. Also exemplary of the sunscreens which may be formulated into the compositions of this invention are physical blockers such as cerium oxides, chromium oxides, cobalt oxides, iron oxides, red petrolatum, silicone-treated titanium dioxide, titanium dioxide, zinc oxide, and/or zirconium oxide, or mixtures thereof.

A wide variety of sunscreens is described in U.S. Pat. Nos. 5,087,445 and 5,073,372, and Chapter VIII of Cosmetics and Science and Technology (1957) by Segarin et al., pages 189 et seq.

Sunscreens which may be formulated into the compositions of the instant invention are those selected from among: aminobenzoic acid, amyldimethyl PABA, cinoxate, diethanolamine p-methoxycinnamate, digalloyl trioleate, dioxybenzone, 2-ethoxyethyl p-methoxycinnamate, ethyl 4-bis(hydroxypropyl)aminobenzoate, 2-ethylhexyl-2-cyano-3,3-diphenylacrylate, ethylhexyl p-methoxycinnamate, 2-ethylhexyl salicylate, glyceryl aminobenzoate, homomenthyl salicylate, homosalate, 3-imidazol-4-ylacrylic acid and ethyl ester, methyl anthranilate, octyldimethyl PABA, red petrolatum, sulisobenzone, titanium dioxide, triethanolamine salicylate, N,N, N-trimethyl-4-(2-oxoborn-3-ylidene methyl)anillinium methyl sulfate, and mixtures thereof.

Sunscreens active in the UV-A and/or UV-B range can also include:
p-aminobenzoic acid,
oxyethylene (25 mol) p-aminobenzoate,
2-ethylhexyl p-dimethylaminobenzoate,
ethyl N-oxypropylene p-aminobenzoate,
glycerol p-aminobenzoate,
4-isopropylbenzyl salicylate,
2-ethylhexyl 4-methoxycinnamate,
methyl diisopropylcinnamate,
isoamyl 4-methoxycinnamate,
diethanolamine 4-methoxycinnamate,
3-(4′-trimethylammunium)-benzyliden-bornan-2-one methylsulfate,
2-hydroxy-4-methoxybenzophenone,
2,4-dihydroxybenzophenone,
2,2′,4,4′-tetrahydroxybenzophenone,
2,2′-dihydroxy-4,4′-dimethoxybenzophenone,
2-hydroxy-4-n-octoxybenzophenone,
2-hydroxy-4-methoxy-4′-methoxybenzophenone,
3-(4′-sulfo)benzyliden-bornan-2-one and soluble salts thereof,
urocanic acid,
2,4,6-tris[p-(2′-ethylhexyl-1′-oxycarbonyl)-anilino]-1,3,5-triazine,
2-[(p-(tertiobutylamido)anilino]-4,6-bis-[(p-(2′-ethylhexyl-1′-oxycarbonyl)anilino]-1,3,5-triazine,
2,4-bis{[4-(2-ethyl-hexyloxy)]-2-hydroxyl-phenyl}-6-(4-methoxy-phenyl)-1,3,5-triazine (“TINOSORB S” marketed by Ciba),
the polymer of N-(2,4)-[(2-oxoborn-3-yliden)methyl]benzyl]-acrylamide,
1,4-bisbenzimidazolyl-phenylen-3,3′,5,5′-tetrasulfonic acid and salts thereof,

the benzalmalonate-substituted polyorganosiloxanes,

the benzotriazole-substituted polyorganosiloxanes (Drometrizole Trisiloxane), dispersed 2,2′-methylene-bis-[6-(2H-benzotriazol-2-yl)-4-(1,1,3,3-tetramethylbutyl)phenol] such as that marketed under the trademark MIXXIM BB/100 by Fairmount Chemical, or micronized in dispersed form thereof such as that were marketed under the trademark TINOSORB M by Ciba Specialty Chemicals Corp. (Tarrytown, N.Y.), and solubilized 2,2′-methylene-bis-[6-(2H-benzotriazol-2-yl)-4-(methyl)phenol] such as that marketed under the trademark MIXXIM BB/200 by Fairmount Chemical. Typically combinations of one of more of these sunscreens are used.

The dibenzoyl methane derivatives other than avobenzone are described, for example, in FR 2,326,405, FR 2,440,933 and EP 114,607.

Other dibenzoyl methane sunscreens other than avobenzone include (whether singly or in any combination):
2-methyldibenzoylmethane
4-methyldibenzoylmethane
4-isopropyldibenzoylmethane
4-tert-butyldibenzoylmethane
2,4-dimethyldibenzoylmethane
2,5-dimethyldibenzoylmethane
4,4′-diisopropyldibenzoylmethane
4,4′-dimethoxydibenzoylmethane
2-methyl-5-isopropyl-4′-methoxydibenzoylmethane
2-methyl-5-tert-butyl-4′-methoxydibenzoylmethane
2,4-dimethyl-4′-methoxydibenzoylmethane
2,6-dimethyl-4-tert-butyl-4′-methoxydibenzoylmethane.

Additional sunscreens that can be used are described in pages 2954-2955 of the International Cosmetic Ingredient Dictionary and Handbook (9^{th} ed. 2002).

The compositions of the present invention may further comprise at least one suitable (e.g., cosmetically or dermatologically acceptable) additive or adjuvant, including, for example, coloring agents (e.g., pigments and dyestuffs), antioxidants, essential oils, preserving agents, fragrances, fillers, pasty fatty substances, waxy fatty substances, neutralizing agents, and polymers, e.g., lipo-soluble polymers.

Examples of preservatives include alkyl para-hydroxybenzoates, wherein the alkyl radical has from 1, 2, 3, 4, 5 or 6 carbon atoms and preferably from 1 to 4 carbon atoms e.g., methyl para-hydroxybenzoate(methylparaben), ethyl para-hydroxybenzoate(ethylparaben), propyl para-hydroxybenzoate(propylparaben), butyl para-hydroxybenzoate (butylparaben) and isobutyl para-hydroxybenzoate(isobutylparaben). Mixtures of preservatives may certainly be used, e.g., the mixture of methyl-paraben, ethylparaben, propylparaben and butylparaben sold under the name Nipastat by Nipa, and the mixture of phenoxyethanol, methylparaben, ethylparaben, propylparaben and butylparaben sold under the name Phenonip, also by Nipa.

In embodiments where the cosmetic compositions are colored due to the presence of at least one pigment, the pigment is preferably treated, e.g., with an amino acid. Treated pigments are known in the art. See, e.g., U.S. Pat. No. 5,843,417. For example, pigments treated with silicones are described in U.S. Pat. No. 4,574,082, and pigments treated with amino acids are described in U.S. Pat. No. 4,606,914. Treated pigments are commercially available from U.S. Cosmetics Corp., a distributor of Miyoshi Kasei (Japan) (e.g., pigments treated with a vegetable-derived amino acid such as disodium stearoyl glutamate, aluminum oxide and optionally titanium dioxide).

Fillers that may be used in the compositions of the invention include silica powder; talc; polyamide particles and especially those sold under the name Orgasol by the company Atochem; polyethylene powders; microspheres based on acrylic copolymers, such as those based on ethylene glycol dimethacrylate/lauryl methacrylate copolymer sold by the company Dow Corning under the name Polytrap; expanded powders such as hollow microspheres and especially the microspheres sold under the name Expancel by the company Kemanord Plast or under the name Micropearl F 80 ED by the company Matsumoto; powders of natural organic materials such as crosslinked or noncrosslinked corn starch, wheat starch or rice starch, such as the powders of starch crosslinked with octenyl succinate anhydride, sold under the name Dry-Flo by the company National Starch; silicone resin microbeads such as those sold under the name Tospearl by the company Toshiba Silicone; clays (bentone, laponite, saponite, etc.) and mixtures thereof.

The amounts of fillers are those conventionally used in the cosmetics field. Generally, the amounts range from about 0.01% to about 20%, and in some embodiments from about 1% to about 10%, relative to the total weight of the composition. Depending on their nature, these adjuvants may be introduced into the oil phase or the aqueous phase.

The composition can be packaged in a suitable container. The choice of container may depend upon the viscosity and intended use of the composition by the consumer. For example, a lotion or fluid cream can be packaged in a bottle or a roll-ball applicator, or a capsule, or a propellant-driven aerosol device or a container fitted with a pump suitable for finger operation. When the composition is a cream, it can simply be stored in a non-deformable bottle or squeeze container, such as a tube or a lidded jar.

The invention will be further described by reference to the detailed examples. These examples are provided for purposes of illustration only, and are not intended to be limiting unless otherwise specified.

### EXAMPLES

### Example 1

### Sunscreen

| | | |
|---|---|---|
| Phase A1: | stearic acid | 1.5% |
| | Triethanolamine | 0.45% |
| | Glycerylstearate/PEG 100 Stearate | 1.5% |
| | Stearyl alcohol | 0.8% |
| | Preservative | Q.S. |
| | Octocrylene | 10% |
| | Avobenzone | 2% |
| Phase A2 | Pemulen TR1 | 0.2% |
| | Isopropyl palmitate | 1.2% |
| Phase A3 | TiO₂ | 2% |
| Phase B1 | humectants (glycerin/propylene glycol) | 15% |
| | EDTA | 0.1% |
| | Preservative | Q.S. |
| Phase B2 | Mexoryl SX | 6% (33% |
| | | aqueous |
| | | solution) |
| | DMEA | 0.89% |
| Phase C | Carbomer 980 | 0.1% |
| | Isopropyl palmitate | 1% |
| Phase D | Cyclopentasiloxane | 7% |
| Phase E | water | QSP 100% |

### Example 2

### Mask

| | |
|---|---|
| Hepes | 10% |
| DMAE | 1.5% |
| Hydroxypropylcellulose | 0.5% |
| Ethanol | 10% |
| Glycerin | 10% |
| Water | QSP 100% |

All publications cited in the specification are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A topical cosmetic composition, comprising:
a mono-hydroxyl substituted amine represented by the formula: 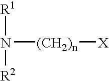
where R¹ and R², which may be same or different, each represents C₁-C₃ alkyl groups, and X represents hydroxyl (OH) and n is 2, 3, 4, or 5, or a cosmetically acceptable salt thereof;
a sulfonic acid derivative, a phosphoric acid derivative, or a phosphonic acid derivative, or a cosmetically acceptable salt thereof;
and a cosmetically acceptable carrier.

2. The cosmetic composition of claim 1, which comprises a sulfonic acid derivative represented by the formula: 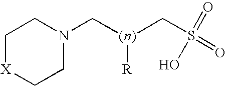
wherein R represents a hydrogen atom, —OH or NH₂; X represents an oxygen atom, a group: 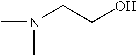
or a group: 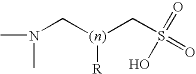
and n is 0, 1, 2, or 3.

3. The cosmetic composition of claim 2, wherein said sulfonic acid derivative is [4-(2-hydroxyethyl)piperazine-1-ethanesulfonic acid].

4. The cosmetic composition of claim 1, which comprises a sulfonic acid derivative which a camphor derivative or a salt thereof.

5. The cosmetic composition of claim 4, wherein said camphor derivative comprises (1,4-divinylbenzene)camphorsulfonic acid.

6. The cosmetic composition of claim 5, further comprising a sunscreen agent other than (1,4-divinylbenzene)camphorsulfonic acid.

7. The cosmetic composition of claim 1, comprising said sulfonic acid which is present in said cosmetic composition in amount about 0.2% to about 30%, based on the total weight of said composition.

8. The cosmetic composition of claim 1, wherein said first active agent comprises a phosphonic acid derivative.

9. The cosmetic composition of claim 8, wherein said phosphonic acid derivative is selected from the group consisting of 1-hydroxy-ethylidene 1,1-diphosphonic acid, aminotri(methylene phosphonic)acid, hexamethylenediaminetetra(methylenephosphonic)acid, diethylenetriamine pentamethylene phosphonic acid; diethylenetriamine tetramethylene phosphonic acid; and ethylenediamine tetramethylene phosphonic acid, and cosmetically acceptable salts thereof.

10. The cosmetic composition of claim 8, wherein said phosphonic acid derivative is present in said cosmetic composition in amount about 0.05% to about 10%, based on the total weight of said composition.

11. The cosmetic composition of claim 1, comprising a phosphoric acid derivative.

12. The cosmetic composition of claim 11, wherein said phosphoric acid derivative is present in said cosmetic composition in amount about 0.05% to about 10%, based on the total weight of said composition.

13. The cosmetic composition of claim 1, wherein said carrier comprises water.

14. The cosmetic composition of claim 1, wherein said cosmetically acceptable carrier comprises a volatile solvent.

15. The cosmetic composition of claim 14, wherein said volatile solvent comprises ethanol

16. The cosmetic composition of claim 13, further comprising a fatty phase.

17. The cosmetic composition of claim 1, further comprising a gelling agent.

18. The cosmetic composition of claim 1, further comprising an active agent selected from the group consisting of sunscreens, anti-acne agents, anti-oxidants, carboxylic acids, and steroidal anti-inflammatory agents.

19. The cosmetic composition of claim 1, wherein said amine is present as a cosmetically acceptable salt.

20. The cosmetic composition of claim 1, 3 or 5, wherein said amine comprises dimethylaminoethanol (DMAE).

21. The cosmetic composition of claim 1, wherein said amine is present in an amount of 0.1% to about 5.0% based on total weight of the said composition.

22. The cosmetic composition of claim 1, wherein said amine is present in an amount of about 0.2% to about 3.0% based on total weight of said composition.

23. A method of treating or conditioning skin, comprising:
applying to skin a cosmetic composition comprising: a mono-hydroxyl substituted amine represented by the formula: 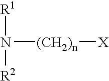
wherein R¹ and R², which may be same or different, each represents C₁-C₃ alkyl groups, and X represents hydroxyl (OH) and n is 2, 3, 4, or 5, or a cosmetically acceptable salt thereof; a sulfonic acid derivative, a phosphoric acid derivative, or a phosphonic acid derivative, or a cosmetically acceptable salt thereof; and a cosmetically acceptable carrier.
